(19) United States
(12) Patent Application Publication
(10) Pub. No.: US 2005/0282225 A1
(43) Pub. Date: Dec. 22, 2005

(54) Quantitative binding assays using green fluorescent protein as a label

(76) Inventors: Sylvia Daunert, Lexington, KY (US); Jennifer C. Lewis, Lexington, KY (US); Emily C. Hernandez, Lexington, KY (US)

Correspondence Address: BIRCH STEWART KOLASCH & BIRCH, PO BOX 747, FALLS CHURCH, VA, 22040-0747, US

(21) Appl. No.: 11/197,429

(22) Filed: Aug. 5, 2005

Related U.S. Application Data
(63) Continuation of application No. 10/765,063, filed on Jan. 28, 2004, now abandoned.
Application No. 10/765,063 is a continuation of application No. 09/042,643, filed on Mar. 17, 1998, now abandoned.

(51) Int. Cl. C12Q 1/68; G01N 33/53

(52) U.S. Cl. 435/6; 435/7.5

(57) ABSTRACT

A heterogeneous binding assay for an analyte in a fluid sample is developed, which uses a green fluorescent protein (GFP) label. A ligand-GFP conjugate has a specific binding affinity for an anti-ligand immobilized on a support. The anti-ligand also has a specific binding affinity for the analyte. Competition between the analyte and ligand-GFP conjugate for binding sites on the anti-ligand permits an assay for an unknown amount of the analyte. Preferred specific binding pairs for use in the assay are biotin:avidin, and a selected antibody and its antigen. A preferred assay employing an antibody and its antigen is illustrated for a fusion protein containing GFP and an antigenic determinant. Picomolar amounts of analyte can be detected. The mutant of GFP that contains a six-histidine tail to facilitate purification on an immobilized metal affinity column is chemically modified to incorporate biotin moieties. The resulting conjugates retain the fluorescence characteristics of the urunodified protein and are used along with avidin-coated magnetic beads in the development of the assay.

### STATEMENT OF GOVERNMENT SUPPORT

Development of the present invention was supported in part by the National Institutes of Health (Grant GM47915) and the Department of Energy (Grant DE-FG05-95ER62010). The Government may have certain rights in the invention.

### TECHNICAL FIELD

The present invention relates to quantitative assays of biomolecules using a fluorescent label. The assays employ a functional green fluorescent protein (GFP) moiety as the label. Preferred aspects of the invention employ the GFP label chemically conjugated to a biotin moiety or fused to an oligo- or polypeptide or an immunoglobulin.

### BACKGROUND OF THE INVENTION

Green fluorescent protein (GFP) is a fluorescent protein that occurs naturally in the jellyfish Aequorea victoria. It has a molecular weight of 27 kDa and is composed of 238 amino acids. GFP has a maximum absorption peak at 395 nm and a minor peak at 470 nm and it emits green light at 507 nm with a shoulder at 540 nm (1) with a quantum yield of 72-80% (2). These fluorescence characteristics are due to the presence of an internal chromophore, formed from the post-translational oxidation of residues Ser⁶⁵, Tyr⁶⁶ and Gly⁶⁷ in the primary structure of the protein (3-4). The crystal structure shows that GFP has a β-barrel structure, with the chromophore residing within the interior of the β-barrel (5). The GFP protein receives its excitation in vivo by radiationless energy transfer from an accessory protein, aequorin (2,6).

In the early 1990s, a gene encoding the natural wild isotype of GFP was cloned, sequenced (7), and expressed in E. coli cells (8). These developments led to efforts to generate mutated forms of GFP in order to alter and occasionally improve its fluorescence characteristics, as well as to render GFP expression possible in a variety of cell types (6,9). The availability of the improved mutants in combination with the excellent natural characteristics of GFP suggest great potential for this protein as a fluorescent marker of biomolecular targets. Apparently, the first application suggested for GFP was as a marker for gene expression in a variety of organisms (10).

The explosion of interest in GFP as a reporter molecule is due to several key advantages over other systems: it operates independently of any cofactors, other proteins, or substrates, thereby making it ideal for living systems; it is stable, species-independent, and resistant to denaturing conditions (alkaline pH, chaotropic salts, organic solvents, and detergents) as well as a variety of proteases; its fluorescence is more stable to photobleaching than fluorescein and it has a high quantum yield; and it is a small, monomeric protein, which permits its incorporation into fusion proteins.

Exemplary of some approaches taken to date for using GFP as a label are the following:

U.S. Pat. No. 5,491,084 and corresponding PCT publication WO 95/07463 to Chalfie et al. propose, among other things, localizing a protein of interest by fusing a DNA sequence encoding the protein to the GFP gene. It is urged that cells transfected with this fused sequence will secrete a fusion protein of the two peptides, thereby affording a method for qualitatively selecting cells expressing the protein of interest.

Several methods of performing cell selection and localizafion of a protein of interest within a cell, e.g., at an organelle, have been described. Wilson, L. et al., Biotechniques, 22: 674-681 (1997) disclose a recombinant method of providing baculoviruses that express the GFP gene, which permits selection of cells transfected with the viruses. Li, Y. et al., Biotechniques, 23: 1026-1029 (1997) disclose the use of GFP as a replacement for the lacZ marker in scoring studies of apoptosis of transiently transfected cells. Li, X. et al., Biotechniques, 24: 52-55 (1998) disclose a method of screening for transfected clones, which employs a bidirectional vector that coexpresses enhanced GFP (EGFP) and a second gene, such as the luciferase gene.

U.S. Pat. Nos. 5,162,227; 5,422,266; 5,541,309 and corresponding EP 187519 to Cormiier and Prasher disclose linking a wild-type apoaequorin gene to a vector and expressing the same in E. coli to produce the aequorin protein. This system differs from the GFP system by employing bioluminescence, rather than autofluorescence, and requires the use of luciferin and Ca²⁺ cofactors.

PCT publication WO 96/27675 of Haseloff et al. discloses a modified GFP, which reportedly affords a more efficient expression in plant cells, e.g., Arabidopsis. A fusion of the modified GFP gene with a nucleotide sequence of interest is also proposed.

PCT publications WO 96/23898 and WO 97/11094 of Thastrup et al. propose the use of a fusion of GFP or a modified GFP with a binding domain of a second messenger or an enzyme recognition site. A method of determining the biological activity of a substance by monitoring the change in fluorescence of the GFP unit is proposed.

U.S. Pat. No. 5,625,048 to Tsien et al. discloses mutant GFP fluorescent proteins and proposes fusions of these proteins with polyhistidine tags to aid in purifying the recombinant proteins.

PCT publication WO 95/19446 of Virta et al. proposes a method of determining the amount of a heavy metal in a sample by monitoring the expression of a luciferase under the control of a promoter sensitive to the heavy metal.

U.S. Pat. No. 5,569,588 to Ashby et al. proposes a method of determining the transcriptional responsiveness of an organism to a candidate drug by detecting the expression of a reporter gene product, such as GFP.

An object of the present invention is to provide a quantitative binding assay that employs GFP as a label. Such an assay is expected to enjoy the many advantages discussed above for GFP. Another object of the invention is to provide quantitative assays based on well studied specific binding pairs, such as biotin for avidin, and immunoglobulins for their respective antigens. A further object of the invention is to develop a heterogeneous binding assay that affords increased sensitivity and/or resolution over homogeneous assays.

### SUMMARY OF THE INVENTION

The present invention is for quantitative biological assays that employ green fluorescent protein (GFP) label. The GFP label can be the native protein or a mutant thereof, such as one having an enhanced intensity of fluorescence.

In one aspect of the invention, an assay can be used to detect picomolar levels of a biotinylated analyte by virtue of the GFP label being chemically linked to a biotin moiety, which is competition with the analyte for binding sites on avidin.

In another aspect, an assay permits detection of picomolar levels of an analyte having an antigenic region, such as an epitope of an antibody described by an oligo- or polypeptide, in an immunoassay. In the immunoassay, a GFP fusion with the oligo- or polypeptide is in competition with the analyte.

In yet another aspect, an assay permits detection of picomolar levels of a binding protein or an immunoglobulin, which competes with a GFP-labeled antibody for epitopes on a common antigen.

In each of the above assays, it is preferred that the binding partner of the analyte is immobilized on a solid support, however, it may alternatively be in solution.

More specifically, a preferred assay of the present invention comprises:
(a) contacting an unknown amount of analyte with a solution having a predefined ratio of a ligand-GFP-conjugate, solution volume, and anti-ligand, in which the anti-ligand is immobilized on a support and has a specific binding affinity for the ligand-GFP conjugate and the analyte;
(b) incubating the analyte with the solution for a predetermined time;
(c) separating the supernatant of the solution from the support;
(d) measuring the intensity of fluorescence of the supernatant; and
(e) relating the measured intensity of fluorescence to the amount of analyte in the sample.

The use of GFP as a label in a quantitative assay of the present invention affords many advantages over previous approaches. Most significantly, GFP does not require any non-ubiquitous cofactors or substrates to exhibit fluorescence. Moreover, the GFP is resistant to heat, detergents, photobleaching, chaotropic salts, and alkaline pH. It is also environmentally safe.

### DESCRIPTION OF THE DRAWINGS

FIG. 1 shows calibration curves for a biotinylated GFP conjugate (●) and unbiotinylated GFP (◯) as described in Example 4.

FIG. 2 depicts a binder dilution curve showing the dependence of fluorescence intensity upon the amount of avidin binder present in the sample as described in Example 5.

FIG. 3 depicts the incubation time response needed for reproducible biotin:avidin binding as described in Example 6.

FIG. 4 depicts a dose response of added biotin (●) in the presence of a fixed amount of biotinylated GFP (3×10⁻⁹ M), and the absence of nonspecific binding of avidin to GFP (◯), as described in Example 7.

FIG. 5 depicts a schematic of the plasmid pSD100 containing the DNA sequences of the octapeptide and the gene of GFP fused in frame.

FIG. 6 depicts a calibration plot of the intensity of fluorescence as a function of the concentration of octapeptide-GFP fusion protein as described in Example 10.

FIG. 7 depicts a binder dilution curve obtained by incubating varying amounts of the M2 antibody immobilized on agarose beads with 100 μL of a 1.1×10⁻⁹ M solution of the octapeptide-GFP conjugate as described in Example 11. Data are the average±one standard deviation (n=3). Some error bars are obstructed by the symbols for the points.

FIG. 8 illustrates the effect of the incubation time on the binding of 100 μL of a 1.1×10⁻⁹ M solution of the octapeptide-GFP conjugate to 200 μL of 140 μg/mL suspension of the M2 antibody immobilized on agarose beads as described in Example 12. Data are the average±one standard deviation (n=3).

FIG. 9 shows a dose-response curve for free, unlabeled octapeptide generated by sequentially incubating 200 μL of 140 μg/mL suspension of immobilized M2 antibody with varying concentrations of the octapeptide for 25 min, followed by incubation with two different amounts of octapeptide-GFP conjugate for an additional 25 min as described in Example 13. In one solution, 90 μL of 1.1×10⁻⁸ M stock solution is added to afford a 5.2×10⁻¹⁰ M solution of the octapeptide-GFP conjugate (▪). In the other solution, 200 μL of the 1.1×10⁻⁸ M stock solution is added to afford a 1.1×10⁻⁹ M solution of the octapeptide-GFP conjugate (♦). Data are the average±one standard deviation (n=3). Some error bars are obstructed by the symbols for the points.

FIG. 10 illustrates the dose independence of octapeptide-GFP binding with M2 antibody in the presence of varying amounts of rev-octapeptide as described in Example 13. The same conditions were used as described for FIG. 9.

### DETAILED DESCRIPTION OF THE INVENTION

A. Definitions

A “ligand”, as used herein, is a solvated molecule that has a specific binding affinity for a particular anti-ligand. Examples of ligands include agonists and antagonists for cell membrane receptors, toxins and venoms, viral epitopes, hormones (e.g., opiates, steroids etc.), hormone receptors, peptides, enzymes, enzyme substrates, cofactors, drugs, lectins, sugars, oligonucleotides, oligosaccharides, proteins, and monoclonal antibodies.

An “anti-ligand”, as used herein, is a molecule having a known or unknown specific binding affinity for a given ligand. An anti-ligand can be immobilized, reversibly or irreversibly, on a surface. An anti-ligands may be naturally-occurring or man-made. Examples of anti-ligands that can be employed by this invention include cell surface receptors, antibodies (polyclonal and monoclonal), antisera reactive with specific antigenic determinants (such as on viruses, cells or other materials), hormones, drugs, oligonucleotides, peptides, enzymes, substrates, cofactors, lectins, sugars, oligosaccharides, cells, cellular membranes, and organelles.

The term “specific binding affinity” refers to the ability of one substance to associate with another substance with a high association constant (>10⁷). The affinity is also one that is specific, i.e., the substances have a binding affinity that discriminates between its preferred binding partner and a non-specifically binding material. An example of a specific binding affinity is that between biotin and avidin, and an example of a non-specific binding affinity is that between two molecules of bovine serum albumin or two identical antibodies.

A “biomolecule”, as referred to herein, is a molecule either naturally occurring in a biological system, or one that can be introduced into a biological system. Examples of such biomolecules include drugs, vitamins, metabolites, and other compounds suspected of having biological activity.

A “green fluorescent protein”, as used herein, refers to a wild-type GFP, as well as any enhanced mutant form, including fragments, substitutions, deletions, homologs and orthologs thereof, which remain functional, i.e., exhibit fluorescence properties.

A “conjugate” as referred to herein refers to a molecule composed of one or more distinct chemical moieties joined together, as through a covalent bond. An example is a ligand-GFP conjugate in which the ligand and GFP units are linked through an amide bond. Another example is a fusion protein in which an oligo- or polypeptide is covalently fused at its C or N terminus to a second oligo- or polypeptide.

A “regulatory sequence” is a DNA sequence necessary for inducing transcription of a gene, and includes a functional promoter and/or enhancer sequence.

The term “operatively linked” as used herein means that a first nucleotide sequence, such as a regulatory element, is fused in frame with a second nucleotide sequence so as to afford a faithful transcription of the entire nucleotide sequence, which upon translation yields the desired protein.

B. Assay Method—General Aspects

An assay of the present invention permits the quantitative determination of an unknown amount of an analyte in a fluid sample either in a heterogeneous or homogeneous setting. A heterogeneous assay is generally preferred due to an enhanced signal thereby provided.

A heterogeneous assay, in which the anti-ligand is immobilized on a solid support, comprises the steps of:
(a) contacting an unknown amount of analyte with a solution having a predefined ratio of a ligand-GFP conjugate, solution volume, and anti-ligand, where the anti-ligand has a specific binding affinity for the ligand-GFP conjugate and the analyte;
(b) incubating the analyte with the solution for a predetermined time;
(c) separating the anti-ligand and any components binding to it from the support to afford a supernatant;
(d) measuring the intensity of fluorescence of the supernatant; and
(e) relating the measured intensity of fluorescence to the amount of analyte in the sample.

In a heterogeneous assay, the solution containing analyte and ligand-GFP conjugate is onveniently separated from the anti-ligand by removing the support from the solution. The physical separation of anti-ligand bound complexes from solution ensures that the measured fluorescence signal is not due in part to anti-ligand complexes.

In an alternative embodiment, the analyte, ligand-GFP conjugate, and anti-ligand are combined in the solution all in the liquid phase, thereby affording a homogeneous assay. In this embodiment, the signal associated with forming binding pairs of ligand and anti-ligand is monitored directly without need to resort to a separation step. However, attenuation of the fluorescence signal upon binding of analyte to anti-ligand can be limited. In this embodiment, separation step (c) is omitted, and the solution containing all components replaces the “supernatant” referred to above. A centrifugation step can be performed to remove any species that interfere with the fluorescence signal if necessary. The fluorescence signal can be enhanced by using evanescent wave fluorimetry if necessary [James, E. et al., Appl. Biochenz. Biotech., 60(3): 189-202 (1996)].

In steps (a) and (b) above, the anti-ligand can be combined with analyte and ligand-GFP conjugate in any order. However, it is generally preferred to incubate analyte with anti-ligand first in order to ensure recognition, followed by incubating the resulting complex with ligand-GFP conjugate, i.e., a “forward” assay.

In a method of the present invention, the aforesaid predefined ratio of ligand-GFP conjugate, anti-ligand and volume is determined by obtaining a binder dilution profile, as illustrated herein in the examples. A method of obtaining such a binder dilution profile is preferably performed with anti-ligand immobilized on a solid support, as part of a heterogeneous assay. A preferred method comprises:
(i) providing a known volume of a solution containing a known amount of ligand-GFP conjugate;
(ii) contacting a known amount of the support and immobilized anti-ligand with the solution;
(iii) incubating the solution containing ligand-GFP conjugate and immobilized anti-ligand for a predetermined time;
(iv) separating the supem atant of the solution from the immobilized anti-ligand;
(v) measuring the intensity of fluorescence of the supernatant;
(vi) repeating steps (i)-(v) for a plurality of known amounts of immobilized anti-ligand; and
(vii) selecting a test amount of immobilized anti-ligand based on the measurements, thereby determining the predefined ratio.

Generally, it is preferred that the selected test amount of anti-ligand is corresponds to the linear region of the curve obtained from the binder dilution profile. For amounts above the plateau region, excess sites are available on the anti-ligand, which precludes setting up an effective competition for binding sites.

Although the above method of obtaining a binder dilution profile is described in terms of a heterogeneous assay, a dilution profile for a homogeneous assay is analogous. That is, the anti-ligand would remain in the liquid phase, and a signal for binding with ligand-GFP conjugate would be monitored directly without a separation step.

In step (e) of the above assay protocol, the relating step entails obtaining a dose response profile. For a heterogeneous assay, this step preferably comprises:
(1) combining a test amount of support having anti-ligand immobilized thereon with a known volume of a solution containing a known amount of ligand-GFP conjugate;
(2) combining a known amount of non-fluorescent ligand with the test amount of support and the solution to form a mixture thereof;
(3) incubating the mixture under predetermined conditions;
(4) separating the supernatant of the mixture from the support;
(5) measuring the intensity of fluorescence of the supernatant;
(6) repeating steps (1)-(5) for a plurality of amounts of the non-fluorescent ligand; and
(7) relating the measurements to the known amounts of non-fluorescent ligand.

For a homogenous assay, the relating step (e) is preferably performed without a separation step, i.e., without step (4). The practicality of such an assay, of course, depends on the availability of a signal dependent upon the amount of added non-fluorescent ligand.

The various incubating steps referred to above are typically performed under the same predetermined conditions. Such conditions as temperature, buffers, cofactors, and the like, are known from manufacturers' protocols and comparable known binding parameters. The incubation time required to ensure meaningful competition in the assay can be determined according to the methods described herein, and is usually 25-30 minutes.

Given the current state of development of GFP mutants, it is expected that still further improved mutants, providing greater fluorescence intensity, stability, and the like, will be developed in the near future. It is contemplated that such “enhanced” GFP labels can be readily incorporated into an assay of the invention. To emphasize this point, it is preferred that an assay employ an “enhanced” GFP, i.e., improved mutant of the native GFP, in a ligand-GFP conjugate of the invention. Enhancement is generally achieved by increasing the extinction coefficient for the excitation wavelength. Preferably, the excitation maxima occurs at about 380-500 nm and the emission maxima occurs at about 450-520 nm. Preferably, the separation between the excitation and emission maxima for a given GFP label is at least about 30-50 nm.

Along these lines, a number of mutant GFPs have been developed, some of which are commercially available. A list of references describing mutant GFPs that have been developed to date in this rapidly evolving area of research is appended hereto in the list of references, and includes acceptable mutant GFPs for use with the present invention. A particularly preferred variant having a 50-100 fold improvement in intensity over the native protein, as well as acceptable excitation and emission profiles, has been recently described [Stauber, R. et al. Biotechniques, 24:462-471 (1998)].

In a preferred embodiment, a ligand is biotin and the corresponding anti-ligand is avidin. Whenever this binding pair is employed in an assay, it is expected that the analyte being measured is a biotinylated biomolecule. Thus, a competition can be established between the biotin-containing analyte and the biotin-labeled GFP fluorophore. Preferably, the avidin source is immobilized on a solid support, e.g., magnetic beads. This permits the amount of analyte in the sample to be determined from the intensity of fluorescence of the supernatant.

Preferred biomolecules for study in an assay of the invention are agonists, antagonists, toxins, venoms, viral epitopes, hormones, hormone receptors, polypeptides, enzymes, cofactors, enzyme substrates, drugs, lectins, sugars, oligonucleotides, oligosaccharides, proteins, and antibodies.

An assay of the present invention can be used in high-throughput screening protocols, as when a large number of samples, e.g., patient specimens, must be analyzed. It should also be apparent that the present invention can be practiced in a multi-analyte context, that is, when it is desired to simultaneously analyze the sample for two or more analytes. In this latter aspect, each of the analytes being studied has the same or similar specific binding affinity as its respective ligand, which is coupled to a distinct GFP label. Discrimination bet veen the different analytes present in the sample is thereby effected by separate monitoring of GFP labels.

For example, multi-analyte screening can involve detection of the levels of A₁ and A₂ in a sample, where A₁ and A₂ represent different analytes. A₁ is in competition with L₁-GFP₁ and A₂ is in competition with L₂-GFP₂. Generally designated, Aᵢ is in competition with Lᵢ-GFPᵢ in such a multi-analyte method, where i represents one of a plurality of species. In these formulas, Aᵢ represents a distinct analyte, Lᵢ represents its corresponding ligand, and GFPᵢ represents a distinct variant of GFP. Computer programs are available to discriminate among a large number of analytes present simultaneously in the sample based on the observed spectral profile.

Another embodiment of the invention is where the analyte to be measured is a non-fluorescent antigen. The analyte is in competition with a ligand that is a hapten immunoreactive with an immunoglobulin. By “immunoreactive” is meant that the immunoglobulin has a specific binding affinity for the hapten. Immunoreactivity can be reasonably assured whenever the hapten is present in an immunogen used to raise the immunoglobulin, as by conventional monoclonal antibody techniques. Typically, the immunogen is made by linking a low molecular weight antigen, less than 2000 daltons, to a larger molecule, e.g., keyhole lymphet (KHL).

In a particularly preferred embodiment, the ligand is covalently linked to the GFP label as a fusion protein. The fusion protein can be obtained directly by chemical coupling of the two molecules. Whenever the moieties are coupled chemically it may be desired to do so in a site-directed fashion, i.e., by coupling the ligand to a selected amino acid of the GFP molecule.

The fusion protein can also be obtained using recombinant DNA techniques, by incorporating a nucleotide sequence encoding the hapten either upstream or downstream of a gene encoding the GFP in an expression vector. The respective nucleotide sequences are, of course, operatively linked in frarne, under the control of one or more regulatory sequences so that the desired fusion protein is expressed in reasonable levels. Suitable cloning vectors are commercially available, as exemplified herein. Transformation and incubation of the expression vector containing the fused genes can be performed according to the manufacturers' suggested protocols.

In yet another aspect of the invention, the measured analyte is a non-fluorescent immunoglobulin or fragment thereof, and the ligand, which is labeled with GFP, is an antibody cross-reactive with the analyte. In this embodiment, the anti-ligand is a hapten for the antibody.

A support for use with the present invention may be biological, nonbiological, organic, inorganic, or a combination of any of these, existing as particles, strands, precipitates, gels, sheets, tubing, spheres, containers, capillaries, pads, slices, films, plates, slides, etc. The support may have any convenient shape, such as a disc, square, sphere, circle, etc. For instance, the substrate may be a polymerized Langmuir Blodgett film, functionalized glass, Si, Ge, GaAs, GaP, silica, silicon nitride, modified silicon, or any one of a wide variety of polymers such as (poly)tetrafluoroethylene, (poly)vinylidenedifluoride, or combinations thereof. Other support materials will be readily apparent to those of skill in the art upon review of this disclosure. In a preferred embodiment the substrate is magnetic glass or polystyrene beads, such as those available from Spherotech (Libertyville, Ill.) or Dynal, Inc. (Lake Success, N.Y.)

If needed, the surface of the substrate can be provided with a layer of crosslinking groups in order to facilitate attachment of anti-ligands to the support. The crosslinking groups are preferably of sufficient length to permit anti-ligands on the surface to interact freely with compounds in solution. Crosslinking groups may be selected from any suitable class of compounds, for example, aryl acetylenes, ethylene glycol oligomers containing 2-10 monomer units, dialdehydes, diamines, diacids, amino acids, sulfhydryls, or hetero-bifunctional combinations thereof. Other crosslinking groups may be used in light of this disclosure. Exemplary preferred crosslinking agents, which form covalent amido linkages with free amino groups, such as those on lysine residues, are glutaraldehyde and dimethylsuberimidate.

The crosslinking groups may be attached to the surface by a variety of methods, which are readily apparent to one having skill in the art. For example, crosslinking groups may be attached to the surface by siloxane bonds formed via reactions of crosslinking groups bearing trichlorosilyl or trisalkoxy groups with hydroxyl groups on the surface of the substrate. Preferably, the crosslinking group used with a glass surface is N-BOC-aminopropyltriethoxy silane. The crosslinking groups may optionally be attached in an ordered array, i.e., as parts of the head groups in a polymerized Langmuir-Blodgett film. The type of crosslinking group selected, and the method selected for attaching it to the surface, will depend primarily on the crosslinking group having suitable reactivity with the anti-ligand, which is desired to be attached to the surface. For example, one method for attaching an anti-ligand to the surface of a surface employs a hetero-bifunctional crosslinking reagent, such as diepoxide, which both activates the surface and provides a group that reacts with an activated binding member. Alternatively, the surface can be activated with cyanogen bromide. Reaction with a binding member containing a terminal amino group permits attachment of the binding member to the surface. (U.S. Pat. No. 4,542,102). In the presence of a carbodiimide or other activating agent, for example, an amine group can be coupled to the carboxyl terminus of a binding member desired to be immobilized on the surface.

Additional length may be added to the crosslinking groups by the addition of single or multiple linking groups. Such linking groups are preferably heterobifunctional, having one end adapted to react with the crosslinking groups and the other end adapted to react with the binding member or another linking group. The linking groups may be attached by a variety of methods readily apparent to one skilled in the art; for instance, esterification or amidation reactions of an activated ester of the linking group with a reactive hydroxyl or amine on the free end of the crosslinking group. A preferred linking group is N-BOC-6-aminocaproic acid (i.e., N-BOC-6-aminohexanoic acid) attached by the BOP-activated ester. After deprotection to liberate the free amine terminus, another N-BOC-aminocaproic linker can be added.

The identification of suitable binding pairs for use with the present invention is facilitated by a consideration of their affinity constants, when known. The affinity constants of some sample classes of compounds suitable for use in the present invention are given in Table 1. Preferably, the affinity constant between the anti-ligand and its binding partner will be greater than about 10⁷ M⁻¹. More preferably, the Ka will be greater than about 10¹⁵ M⁻¹ and most preferably, the Ka will be about 10¹⁵ M⁻¹ or greater.

TABLE 1

| Binding Pair | Ka (M⁻¹) |
|---|---|
| Membrane sites: Lectins | 10⁶⁻⁷ |
| Haptens: Antibodies | 10⁵⁻¹¹ |
| Biotin: Avidin | 10¹⁵ |
| Iminobiotin: Avidin | 10¹¹ |
| 2-thiobiotin: Avidin | 10¹³ |
| Dethiobiotin: Avidin | 10¹³ |
| 3′-N-methoxy- | 10⁹ |
| carbonylbiotin methyl ester: Avidin | |

*References: U.S. Pat. No. 4,282,287; Green, “Avidin” in Advances in Protein Chemistry, Academic Press, vol. 29, 105 (1975).

C. Assays Employing a GFP Label Linked to Biotin

Biotin, also referred to as vitamin H, is a 244 Da non-peptidyl molecule that tightly binds to avidin and streptavidin. GFP can be chemically conjugated with biotin using conventional techniques to give a biotinylated GFP molecule of the present invention. Some commercial sources of isolated GFP are currently available, however, they tend to be very expensive. Therefore, it is desired to express GFP from E. Coli that have been transformed with a plasmid encoding the protein. A preferred mode of expressing GFP in this manner is to transform E. Coli with a plasmid encoding GFP immediately downstream of a polyhistidine tail. The resulting polyhistidine-GFP protein can be purified using immobilized metal ion affinity chromatography. The purified protein can then be chemically biotinylated using a long chain derivative of sulfo-NHS-biotin. Due to the electronic isolation of the GFP fluorophore, the biotinylated GFP shows essentially the same excitation and emission maxima as native GFP. Similarly, a biotinylated mutant of GFP has essentially the same excitation and extinction profile as the free mutant GFP protein. As discussed hereinafter, detection limits of 4×10⁻⁹ M biotin, which corresponds to 6.2 pmol biotin, can be obtained using avidin-coated magnetic beads.

A biotin labeled GFP compound of the present invention strongly binds to a variety of avidin species. Many avidin reagents are commercially available, e.g., from Vector Laboratories, Inc. (Burlingame, Calif.). Avidin obtained directly from egg whites has relatively nonspecific binding properties. Therefore, purified avidin products such as Avidin D™, Avidin DN™ (for binding nucleic acids), and Avidin DX™ (for binding to solid supports where relatively nonspecific binding can be tolerated), may be preferred.

In a preferred embodiment, the desired source of avidin is applied to a solid support, which facilitates separation of a bound biotin-labeled product from solution. One recently developed approach is to coat magnetic beads with avidin, which permits separation of the desired biomolecule from solution upon application of an external magnetic field. Superparamagnetic beads covalently coupled to streptavidin are commercially available from Dynal, Inc. (Lake Success, N.Y.).

A combined avidin:biotin:GFP (ABG) marker complex is also contemplated for use in the invention. In this application, a biotinylated biomolecule of interest can be contacted with a previously prepared ABG complex, where the BG component of the complex is prepared according to the methods of the present invention. The subject biomolecule is thereby visualized with the GFP label using an intermediary, highly selective biotin:avidin:biotin “linker”.

An instant biotin-GFP complex can be used to determine concentration levels of a biomolecule linked to a secondary biotin label. Biotin can be used to label a wide variety of biomolecules of interest. For example, levels of biotinylated immunoglobulins and fragments thereof, biotinylated protein A (which is immunoreactive with immunoglobulins), biotinylated lectins, biotinylated anti-lectins, and many other proteins of interest can be identified. Many of these products are commercially available or can be prepared on a custom basis, e.g., from Vector Laboratories, Inc. (Burlingame, Calif.).

Analogs of biotin can also be used in place of native biotin. Exemplary of such analogs are dethiobiotin, iminobiotin, 2-thiobiotin, azabiotin, biocytin and biotin sulfone. Other biotin analogs include those discussed in N. Green, “Avidin” in Advances in Protein Chemistry, Vol. 29, Academic Press, pp 85-133, 1975. Still other biotin analogs, including those obtained more recently, are readily identifiable by the skilled practitioner.

Biotin or a biotin analog can be conjugated to an analyte to be assayed, or to GFP, either directly or by employing a biotinylating reagent. A biotinylating reagent, such as sulfo-NH-LC-biotin, is preferred. Alternatively, the free carboxyl group of biotin can be coupled to a free amino group of the analyte or GFP using a carbodiimide, as is well-known.

D. Assays Employing Translated GFP as Label

Another aspect of the invention entails labeling an oligo- or polypeptide with GFP using recombinant DNA techniques. In particular, the gene encoding GFP is fused to a nucleotide sequence encoding the polypeptide of interest in a suitable expression vector. Upon expressing the vector in a suitable host, and optionally following a purification protocol, the desired GFP fusion protein can be isolated. The isolated GFP labeled protein can then be used in a competitive binding assay in which the labeled protein is in competition with free polypeptide for binding with immunoglobulins specific for the polypeptide. The detection limit for a competitive binding assay of this type is on the order of 1×10⁻⁹ M, which corresponds to sub-picomole levels of analyte.

Recombinant techniques for preparing a fusion protein as described are described, e.g., by Maniatis, T., et al., Molecular Cloning A: Laboratory Manual; Cold Spring Harbor Laboratory Press: Cold Spring Harbor, N.Y., 1989.

Oligonucleotides can be synthesized directly, e.g., using the phosphoramidite method [Itakura, K. et al., Ann. Rev. Biochem., 53:323-356 (1984)], or by PCR amplification of a nucleotide sequence from a genomic or cDNA clone. The PCR method is the subject of U.S. Pat. Nos. 4,683,202 and 4,683,195, and 4,889,818. Direct cloning of PCR generated DNA into a cloning vector is the subject of U.S. Pat. No. 5,487,993. The disclosures of these patents are incorporated herein by reference. Commercial suppliers of suitable expression vectors provide protocols that can be used to operatively incorporate the DNA into the vector.

Methods for carrying out the transformation of a suitable cell line, e.g., E. Coli, with a vector encoding a fusion protein of the invention are described in Maniatis, T., et al., Molecular Cloning A: Laboratory Manual; Cold Spring Harbor Laboratory Press: Cold Spring Harbor, N.Y., 1989. The selection of transformed colonies, e.g., with hybridization techniques, as well as the conditions and protocols for expressing the desired protein, are described in the same reference. Purification methods are straightforward in view of the present disclosure and preferably utilize the instant binding pairs.

Methods for preparing antibody preparations, e.g., polyclonal antibodies, are well known. More preferred are monoclonal antibodies in view of their enhanced selectivity. A method of preparing and isolating monoclonal antibodies is described by Kohler and Milstein, Nature, 256: 495-497 (1975).

E. Assays Employing GFP Chemically Conjugated to an Immunoglobulin or Binding Protein

A still further aspect of the invention entails employing a GFP-labeled antibody preparation or antibody fragment in a competitive assay for the same or similar antibody preparation or fragment. Polyclonal and monoclonal antibodies can be prepared and isolated as described hereinabove. In this embodiment the ligand is represented by an antibody or fragment and the anti-ligand is a hapten for the antibody. The analyte is represented by a non-fluorescent antibody or fragment, which effectively competes for binding sites on the hapten.

The antibody or fragment can be covalently linked to GFP by chemical techniques. Coupling through a terminal or side chain amino, carboxyl, or sulfhydryl group is preferred.

The contemplated protocols for performing an instant antibody-based assay are several in number. As mentioned, an assay preferably employs monoclonal antibody preparations. The assay protocols can be of the forward, fast forward, reverse, or simultaneous types. These different methods are the subject of U.S. Pat. No. 4,376,110, the disclosure of which is incorporated herein by reference. These various assays all involve presenting an antigen in solution so that it is recognized by an antibody or antibody fragment. A preferred method of providing the antigen on a solid matrix entails a “sandwich” approach in which the antigen binds to two antibodies, with one of the antibodies affixed to the surface of the solid matrix. The free valence of the antigen is then available for competition with one or more antibodies.

Similarly, a non-immunoglobulin binding protein can be employed in a competitive assay for the same or similar protein. The binding protein has a specific binding affinity for its binding partner. One binding protein discussed previously is avidin, which binds to biotin. Another class of binding protein contemplated by the present invention includes extra- and intracellular receptors, which bind to their respective ligands, e.g., as part of a signaling pathway. The list of possible receptors is too extensive to enumerate, however, it includes such binding pairs as glucose for its receptor, insulin for its receptor, dopamine for its receptor, and so on. The binding protein substitutes for antibody in the above-described method, and the substrate of the binding protein (food molecule, hormone, neurotransmitter, and the like) substitutes for ligand. It should be appreciated that either the binding protein or its ligand can be immobilized on a solid matrix and used in an assay according to the principles of the present invention.

The invention will now be described with reference to certain examples, which illustrate but do not limit it.

### EXAMPLES

Materials and Apparatus.

An electrophoresis PhastSystem from Pharmacia LKB (Uppsala, Sweden) was used to determine all protein purities through sodium dodecyl sulfate polyacrylamide gel electrophoresis (SDS-PAGE). Dialysis was carried out using Spectra/Por 7 membranes with a molecular weight cut-off of 3,500 from Fisher Scientific (Pittsburgh, Pa.). A magnetic tray from Corning (Walpole, Mass.) was used for all separation steps involving magnetic beads. A Hewlett Packard 8453 UV-visible ChemStation (Wilmington, Del.) was used for all absorbance measurements, while a Mineralight multiband UV 254/366 nm lamp UVP (Upland, Calif.) was used for visual identification of the protein. All fluorescence measurements were obtained in a Fluorolog-2 spectrofluorometer from Spex Industries (Edison, N.J.) using methacrylate disposable cuvettes (Fisher Scientific). Polymerase chain reactions were carried out on a Perkin Elmer GeneAmp PCR System 2400 (Norwalk, Conn.). Visual identification of the protein employed a Mineralight multiband UV 254/366 nm lamp UVP (Upland, Calif.).

Luria-Bertani broth (LB broth) and isopropyl-thio-β-galactopyranoside (IPTG) were obtained from Gibco-BRL (Gaithersburg, Md.) or Difco Laboratories (Detroit, Mich.). Ampicillin (amp), imidazole, tris(hydroxymethyl)aminomethane (Tris), ethylenediaminetetraacetate (EDTA) sodium salt, dithiothreitol (DTT), bovine serum albumin (BSA), agar, glucose, sodium dodecyl sulfate (SDS), and all other reagents were purchased from Sigma (St. Louis, Mo.). Aqueous solutions were prepared using distilled water that was deionized with a Milli-Q water purification system from Millipore (Bedford, Mass.). All chemicals were reagent grade or better and were used as received.

E. coli strain JM109 was obtained from Promega (Madison, Wis.). Green fluorescent protein, plasmid p6xHisGFP and TALON™ resin were purchased from CLONTECH Laboratories (Palo Alto, Calif.). The bicinchoninic acid (BCA) protein assay kit and sulfo-N-hydroxysuccinimide-long chain-biotin (sulfo-NHS-LC-biotin) were purchased from Pierce Chemical (Rockford, Ill.). Streptavidin-FITC was obtained from Vector Laboratories (Burlingame, Calif.). Sphero avidin-coated magnetic particles of 1.0 to 2.0 μm diameters (VMX-10⁻¹⁰) were obtained as a 0.5% w/v suspension from Spherotech (Libertyville, Ill.).

The pFLAG-ATS vector and the anti-FLAG M2 antibody immobilized on agarose beads (2.8 mg/mL suspension) were obtained from IBI-Kodak (New Haven, Conn.). The unlabelled octapeptide (Asp-Tyr-Lys-Asp-Asp-Asp-Asp-Lys), the reverse octapeptide (rev-octapeptide, Lys-Asp-Asp-Asp-Asp-Lys-Tyr-Asp), and all primers used for polymerase chain reaction (PCR) were synthesized and purified by the University of Kentucky Macromolecular Center. The pGFP cDNA vector was purchased from CLONTECH (Palo Alto, Calif.).

I. GFP Label Linked to Biotin

### Example 1

### Expression of 6xHisGFP

The 6xHisGFP protein was expressed in E. coli from plasmid p6xHisGFP by employing a slightly modified protocol from that suggested by the manufacturer. A stock LB-amp plate was prepared and kept refrigerated until use. A colony was picked from the stock plate and inoculated into 20 mL of LB-amp media (100 μg/mL ampicillin). After incubation at 37° C. for 6 h, the broth was transferred to fresh LB-amp media and allowed to grow overnight at 37° C.

Then, IPTG was added to make its concentration in the media 1 mM, and the cells were allowed to grow overnight at 37° C. The cells were centrifuged, and the cell pellet was kept at −80° C. until further use.

### Example 2

### Purification of 6xHisGFP

The cell pellet was thawed at room temperature and resuspended in 50 mM NaH₂PO₄, 10 mM Tris-HCl, 500 mM NaCl, pH 8.0 buffer (buffer A). The cells were sonicated, centrifuged, and the supernatant containing the protein was reserved as the crude cell lysate. The TALON™ resin was thoroughly washed and equilibrated in buffer A. The crude cell lysate was added and incubated for 20 min at room temperature with mild shaking. After four washings with buffer A, the resin was resuspended and packed into a column. Two washes with buffer A and two with buffer A containing 5 mM imidazole followed. The protein was eluted with 500 μL aliquots of elution buffer (buffer A+50 mM imidazole). The resin was washed with deionized water and stored in 20% (v/v) ethanol, 0.1% (w/v) sodium azide solution. The collected fractions were illuminated with long-wavelength UV light, and those that exhibited green fluorescence were reserved.

The purity of the fractions was verified by SDS-PAGE. The concentration of protein was determined using BCA analysis with bovine serum albumin (BSA) as the standard after extensive dialysis against 0.1 M sodium bicarbonate, pH 8.2, buffer (assay buffer).

### Example 3

### Preparation and Characterization of Biotinylated 6xHisGFP Conjugates

A volume of 2 mL of 5×10⁻⁷ M 6xHisGFP in assay buffer was placed in a vial and enough solid sulfo-NHS-LC-biotin was added to result in a 10,000:1 (mole:mole) ratio. The reaction was conducted for 24 h at 4° C. with mild stirring. Extensive dialysis against assay buffer followed. The concentration of each biotinylated 6xHisGFP conjugate (b-6xHisGFP) was determined by BCA analysis using BSA as the standard. The degree of conjugation was determined by a modification of a previously described protocol (11). A volume of 30 μL of each conjugate was placed in separate vials with 300 μL of 6 M HCl. Each vial was frozen with liquid nitrogen and vacuum sealed. A hydrolysis reaction was conducted for 24 h at 100° C. After completion, the vials were opened and the contents transferred quantitatively to centrifuge tubes and spin dried completely (˜6 h) using a DNA Speed Vac 110 vacuum centrifuge from Savant Instruments (Farmingdale, N.Y.). The resulting solid was redissolved in 500 μL of assay buffer. A 4 mg/L solution of streptavidin-FITC in assay buffer and biotin standards ranging in concentration from 10⁻⁶ to 10⁻⁸ M were freshly prepared. A calibration plot for biotin was produced by measuring the enhancement in fluorescence intensity of streptavidin-FITC upon addition of the biotin standards (excitation at 495 nm, emission at 518 nm). The samples containing the hydrolyzed conjugates were treated in the same manner, and the corresponding fluorescence enhancement was measured as well. The concentration of biotin in the unknown samples was determined by interpolation.

### Example 4

### Calibration Curvefor 6xHisGFP and b-6xHisGFP

Solutions ranging in concentration from 5×10⁻⁸ to 1×10⁻¹² M of 6xHisGFP or of b-6xHisGFP were prepared by serial dilutions in assay buffer. The intensity of fluorescence emission at 507 nm was measured upon excitation at 395 nm.

### Example 5

### Binder Dilution Study

A 10:1 dilution of avidin-coated magnetic beads in assay buffer (working dilution) was prepared. Variable volumes of beads (0 to 500 μL) were placed in glass tubes. The storage solution of the magnetic beads was removed by placing the tubes in the magnetic tray after which the supernatant was discarded. The beads were rinsed by adding 500 μL of assay buffer, placing the tube in the magnetic tray and discarding the supernatant. This procedure was repeated six times. After the rinses, 500 μL of a 9×10⁻⁹ M b-6xHisGFP solution was placed in each tube, for a final concentration of conjugate after dilution of 3×10⁻⁹ M. After incubating for 30 min, the tubes were placed back in the magnetic tray and 500 μL of the supernatant was carefully transferred to plastic cuvettes. An additional 1 mL of assay buffer was added to each cuvette. The intensity of fluorescence emission at 507 nm was measured.

### Example 6

### Incubation Time Study

A volume of 75 μL of working dilution of magnetic beads was placed in each tube. The storage solution was removed as described above and the beads were rinsed three times with assay buffer. A volume of 500 μL of a 9×10⁻⁹ M b-6xHisGFP solution was added, for a final concentration of conjugate after dilution of 3×10⁻⁹ M. The tubes were incubated for variable time (0 to 60 min) and returned to the magnetic tray. The remaining procedure was as above.

### Example 7

### Dose-Response Curve for Biotin

A volume of 75 μL of the working dilution of magnetic beads was placed in glass tubes. The storage solution was removed and the beads were rinsed three times with assay buffer. Then, 500 μL of solutions containing both b-6xHisGFP and biotin were added to each tube. The b-6xHisGFP was present at a fixed concentration (1.8×10⁻⁸ M) while biotin was present at concentrations ranging from 6×10⁻⁶ to 6×10⁻¹⁰ M to result in final concentrations after dilution of 3×10⁻⁹ M b-6xHisGFP and 1×10⁻⁶ to 1×10⁻¹⁰ M biotin. The samples were incubated for 30 min and the rest of the procedure was as above.

II. Fusion with Translated GFP Label

### Example 8

### Preparation and Isolation of Octapeptide-GFP Fusion

The gene sequence of GFP was amplified by PCR from the GFP expression vector, pGFP cDNA, using the following flanking primers:

5′-gcggcggcgaagcttatgagtaaaggagaagaacttttc-3′
(SEQ ID NO: 1) and
5′-gcggcggcgaagcttctatgtatagttcatccatgcg-3′
(SEQ ID NO: 2).

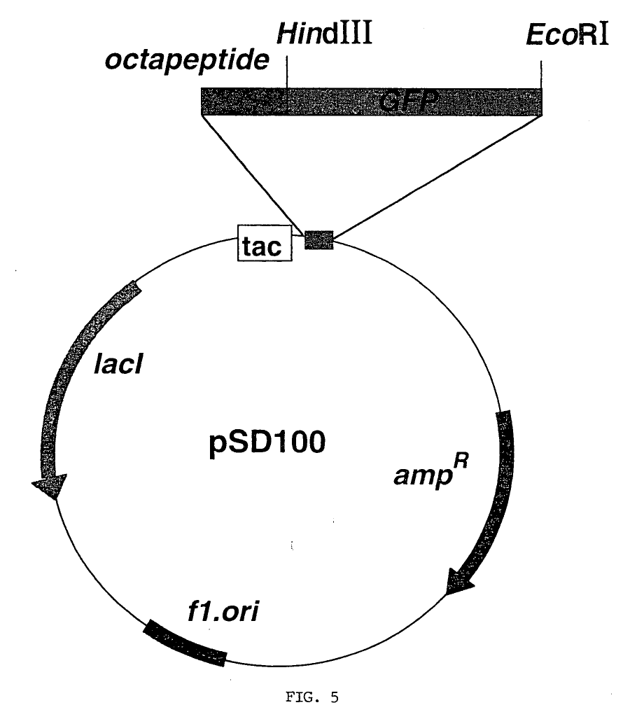

The amplification reaction was carried out using pfu polymerase in a total volume of 50 μL including 250 μM of each dNTP, 25 pmol of each primer, and 1 unit of the polymerase. Thermal cycling parameters were 94° C. for 30 s; 50° C. for 30 s; 72° C. for 1 min 30 s, for 30 cycles. The product was introduced into the multiple cloning site of the pFLAG-ATS vector as a HindIII-EcoRI fragment, to yield the pSD 100 vector, which contains the DNA sequence that codes for the octapeptide-GFP fusion protein (FIG. 5). E. coli strain JM109 was transformed with the pSD 100 vector and then cultured to express the fusion protein. Specifically, the bacteria were grown in 500 mL of LB broth containing ampicillin (50 μg/mL) for 24 h at 37° C. and then an additional 12 h at room temperature. The fusion protein was expressed in the cytoplasm and was isolated as a whole cell extract using lysozyme, followed by centrifugation and collection of the supernatant according to the manufacturer's specifications. All molecular biology procedures were performed using standard protocols [Maniatis, T. et al. Molecular Cloning. A Laboratory Manual; Cold Spring Harbor Laboratory Press: Cold Spring Harbor, N.Y. (1989)].

### Example 9

### Purification of the Octapeptide-GFP Fusion

The crude supernatant obtained from above was mixed with the required amounts of Tris and sodium chloride, and the pH was adjusted using HCl to obtain a solution that was 50 mM Tris-HCl, 150 mM NaCl, pH 7.4 (TBS). The solution was loaded onto an Anti-FLAG M2 affinity column pre-equilibrated with the previous buffer. The column was washed three times with the TBS buffer. The protein was eluted with 6×1 mL aliquots of 0.1M glycine at pH 3 into vials containing 1M Tris base at pH=8.0. The fractions containing the protein were easily determined by using a long wavelength handheld UV lamp. The purity of the octapeptide-GFP conjugate was verified by SDS-PAGE on 12.5% polyacrylamide PhastGels (Pharmacia Biotech, Piscataway, N.J.), which were developed by silver staining (Development Method 210, Pharmacia Biotech). The protein concentration was estimated by using the BCA protein assay, with BSA as the standard.

### Example 10

### Calibration Plots for Octapeptide-GFP Fusion and Binder

In order to perform a calibration plot, a stock solution of the octapeptide-GFP conjugate was serially diluted with a phosphate-buffered saline (PBS) solution (0.10 M NaH₂PO₄, pH 7.4, and 0.15 M NaCl). A calibration plot was prepared by measuring the fluorescence emission intensity at 509 nm using an excitation wavelength of 395 nm.

### Example 11

### Binder Dilution Study

The M2 antibody immobilized on agarose beads was serially diluted from a stock suspension of 2.8 mg of immobilized antibody per milliliter of suspension, and incubated with the octapeptide-GFP conjugate for 60 min (200 μL of each dilution of beads, 1.7 mL of PBS and 100 μL of 1.1×10⁻⁹ M of octapeptide-GFP). After the incubation, the beads were pelleted down by centrifuging the test tubes at 2000 rpm for 15 min at room temperature. Then 1.7 mL of the supernatant was placed into the disposable fluorimeter cuvettes for measurement as above. This process was repeated three times. A volume of 1.7 mL of PBS buffer was used as a blank.

### Example 12

### Incubation Time Study

The optimum time of incubation for the assay was determined by incubating 200 μL of a 140 μg/mL suspension of the immobilized antibody with 100 μL of 1.1×10⁻⁹ M of octapeptide-GFP conjugate in 1.7 mL of PBS buffer for various times.

### Example 13

### Dose-Response Curve and Selectivity Studies

A dose-response curve was constructed by incubating 200 μL of a solution of different concentrations of the octapeptide with 200 μL of a 140 μg/mL suspension of the immobilized antibody and 90 μL of 1.1×10⁻⁸ M of octapeptide-GFP conjugate and 1.510 mL of PBS buffer.

The incubations were performed in a sequential manner. Initially, the octapeptide was incubated with the beads for 25 min. Then, the tubes were centrifuged at 2000×g for 15 min, the supernatant was removed, and the beads were washed three times with PBS. PBS (1.710 mL) and the octapeptide-GFP conjugate were added, and the mixture was incubated for an additional 25 min at room temperature. The beads were then centrifuged again as described, and 1.7 mL of the supernatant was transferred to plastic cuvettes for measurement of fluorescence emission at 508 nm. The selectivity of the assay was evaluated by constructing a dose-response curve for rev-octapeptide, an octapeptide with the amino acids of the octapeptide in the reverse order.

Results and Discussion

Biotin Assays

This work explores the feasibility of using the naturally fluorescent GFP as a label in binding assays. Biotin is used as a model analyte while avidin-coated magnetic beads are employed as the binder. It should be appreciated that other analytes, e.g., those conjugated with biotin, can also be employed in an assay of the invention. Likewise, analogs of biotin can be employed, either alone or as a conjugate, in the assay. As shown, GFP has excellent physical and fluorescence characteristics such as a high fluorescence quantum yield, independence from external cofactors, and high stability to extremes of pH, temperature, and chaotropic agents. These satisfy the requirements for a rugged, safe, and efficient fluorophore to be used as a label in assay development.

Unlike phycobiliproteins that have been used in binding assays (12, 13), GFP is much easier to modify by genetic means (9). For example, studies have been performed to alter the fluorescence characteristics of GFP in terms of the wavelength of excitation and/or emission. Although the goal of these studies was to enable researchers to follow gene expression, these GFP mutants could be highly beneficial in binding assays where more than one analyte need to be determined simultaneously. In such cases, it has been customary to use two different types of labels (e.g., glucose-6-phosphate dehydrogenase and malate dehydrogenase) (14): Therefore, the ability to use two different mutants of the same protein, GFP, in such assays, should greatly simplify the overall assay. Finally, the use of a GFP fusion protein that contains a six-histidine tail at the N-terminus of the protein facilitates purification by using immobilized metal ion affinity chromatography (IMAC) (15).

The 6xHisGFP protein was expressed in E. coli from plasmid p6xHisGFP. The six-histidine tail at the N-terminus of GFP has high affinity for the cobalt on the IMAC column. To discriminate against other proteins that may interact with the IMAC column through their histidine residues, the column was washed with a 5 mM imidazole-containing buffer. Imidazole competes with the histidines for coordination to the immobilized metal and causes elution of the proteins that are associated to the column in a weaker manner. This 5 mM concentration of imidazole, however, does not cause elution of the 6xHisGFP. The latter is eluted when a buffer containing 50 mM imidazole is passed through the column.

The isolated protein was chemically biotinylated with sulfo-NHS-LC-biotin. In particular, three different preparations of 6xHisGFP were biotinylated using the same reaction conditions. The degrees of biotinylation of the resulting conjugates were 0.82, 1.84 and 1.75. It should be noted that the degree of biotinylation was calculated after complete hydrolysis of the conjugate, followed by analysis of the released biotin. Therefore, the degree of biotinylation reflects the total number of biotin molecules attached to the 6xHisGFP. This is a low conjugation ratio, especially when considering that GFP has twenty lysines plus the N-terminus that, if accessible, should provide sites of attachment for biotin. Usually, low degrees of conjugation are achieved by using a low ratio of biotinylating reagent to protein, short reaction times, and careful control of the reaction pH (16). In our studies, even when a high biotinylating reagent to protein ratio (10,000:1, mole:mole) and a long reaction time (24 h) were employed, the degree of conjugation obtained was still less than 2. A possible explanation for the low degree of biotinylation obtained could be related to structural features of the protein. GFP stability is rationalized by the presence of a complex network of hydrogen bonds covering the surface of the β barrel. While this protects the protein from attack by proteases and other environmental threats, it may also tie up lysines that would otherwise be available for biotinylation. Incidentally, the low degree of biotinylation is advantageous when developing binding assays (17-19).

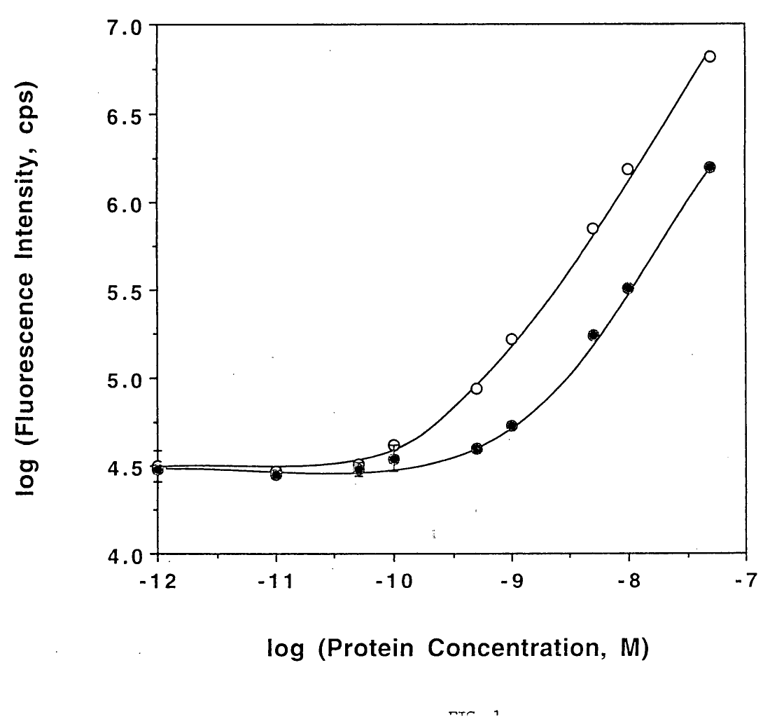

The b-6xHisGFP conjugates retained the same fluorescence excitation and emission wavelengths as 6xHisGFP, but with significant differences in intensity. Representative calibration curves for one of the conjugates and the unbiotinylated protein are shown in FIG. 1. Both proteins exhibit an extended linear range. In terms of detection limits, there is a shift towards worse values for the b-6xHisGFP conjugate. This is related to the lower fluorescence intensity of the biotinylated protein as compared to the 6xHisGFP. The detection limits of each of the three conjugates were evaluated by constructing calibration plots in triplicates. The detection limit was calculated to be 4.05×10⁻¹⁰ M (average of 9 data points from three different conjugates). The pool standard deviation for the detection limit was 0.13×10⁻¹⁰ M. The detection limits were calculated using the average plus three times the standard deviation of the blank (20).

In selecting the concentration of conjugate to develop the assay, a tradeoff between the amount of protein used and the magnitude of signal needs to be considered. The goal is to work with as large a signal as possible, thus maximizing sensitivity and reproducibility of the assay, while using a low concentration of conjugate to assure low detection limits. Therefore, a concentration of 3×10⁻⁹ M of b-6xHisGFP was selected as the working concentration.

Using this concentration of b-6xHisGFP, the effect of avidin on the fluorescence of the conjugate was studied. Additions of avidin in solution in concentrations ranging from 10⁻¹⁰ to 10⁻⁶ M resulted in no significant change in the fluorescence characteristics of the conjugate. Because of the high affinity of avidin to biotin as well as the depth of the binding pocket of avidin, it has been previously observed that avidin can cause changes in the catalytic properties of biotinylated enzymes (21,22). The lack of change in the fluorescence properties of biotinylated GFP when bound to avidin in solution can be rationalized by the location of the chromophore inside the β-barrel structure of GFP, that protects its integrity from environmental factors, including the binding of a large biomolecule like avidin. This is advantageous in the development of heterogeneous competitive binding assays because the interaction between the two biomolecules does not result in a loss in signal. Therefore, the use of avidin-coated magnetic beads to develop the assay was examined.

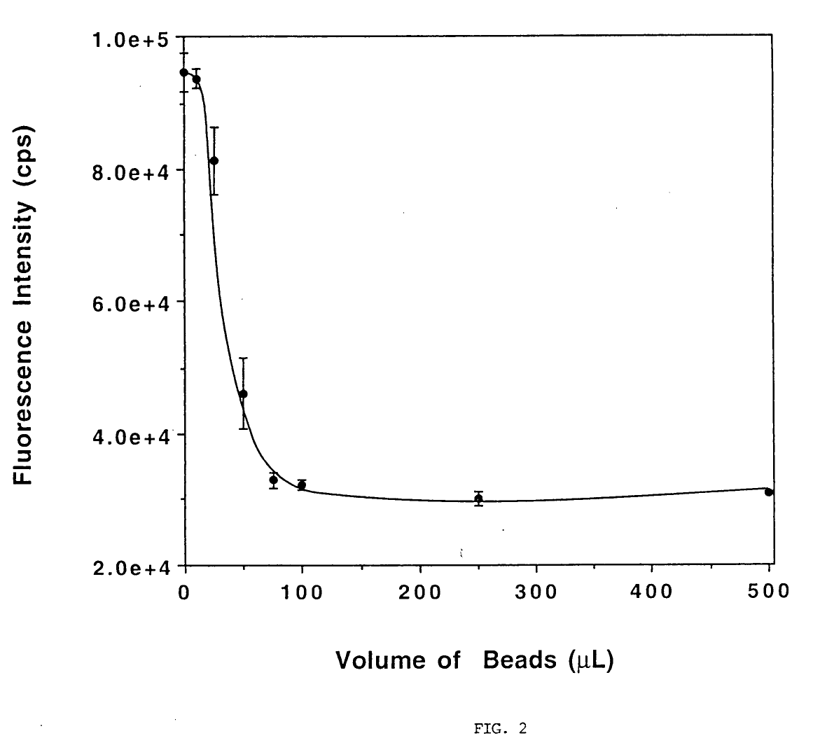

In order to identify the amount of binder to be used in the development of the assay, a binder-dilution curve was prepared by fixing the concentration of conjugate (3×10⁻⁹ M) and adding varying amounts of avidin-coated beads. After incubation for 30 min, the solid and liquid phases were separated and the fluorescence intensity in the supernatant was measured at 507 nm after excitation at 395 nm. The binder-dilution curve (FIG. 2) shows that at low binder concentrations only a small amount of b-6xHisGFP is bound to the solid phase, resulting in a large fluorescence signal in the liquid phase. After 75 μL of beads, the fluorescence intensity reaches a plateau, indicating that all the b-6xHisGFP present is bound to the beads. We selected 75 μL as the volume of beads for the development of the assay.

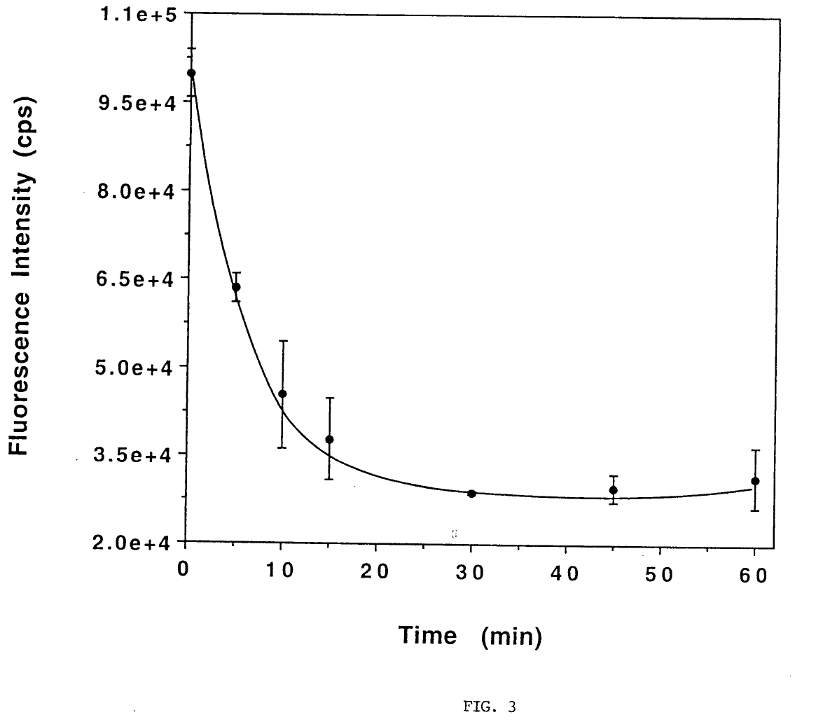

The incubation time needed for reproducible binding was also studied, and the results are shown in FIG. 3. The curve exhibits a plateau at higher incubation times, with equilibrium reached at an incubation time of 30 min. Therefore, 30 min was selected as the optimized incubation time.

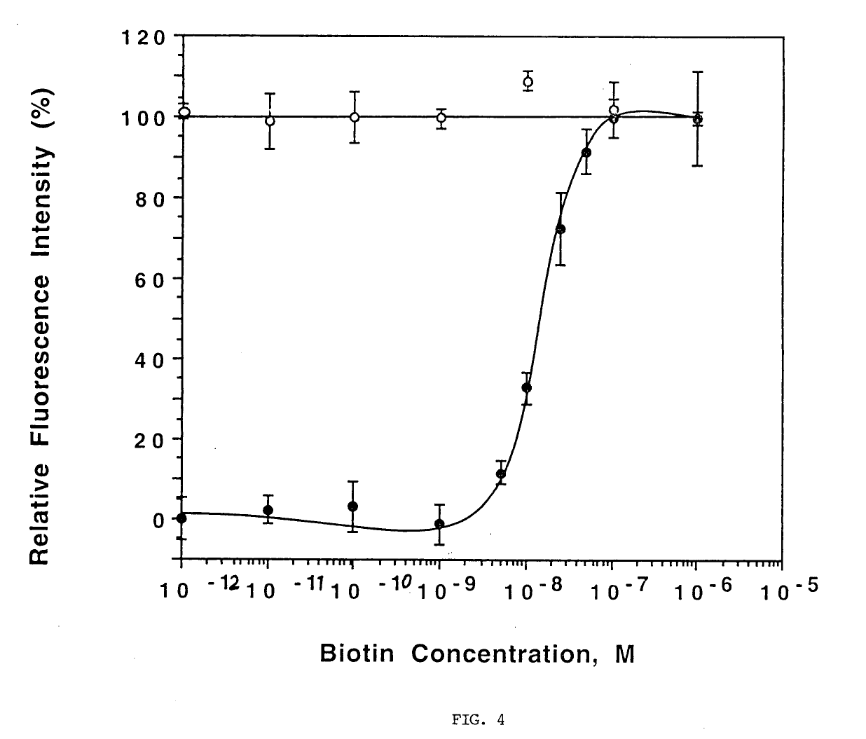

Using the optimized parameters determined (3×10⁻⁹ M b-6xHisGFP, 75 mL of beads and 30 min incubation time), a dose-response curve for biotin was constructed (FIG. 4). Since both the concentrations of binder and conjugate are kept constant during the assay, the observed response is solely dependent on the changes in biotin concentration in the sample. At high concentrations of biotin, the binding sites of avidin become saturated, leaving the conjugate unbound and free in the liquid phase, resulting in a high fluorescence signal. On the other hand, at low biotin concentrations, the sites in avidin are available to be occupied by the conjugate, depleting the liquid phase and resulting in a low fluorescence signal. This gives a sigmoidal shaped curve that exhibits detection limits for biotin of (1.04±0.04)×10⁻⁸ M, determined as described above (20). To verify that the binding observed is due to the presence of biotin in b-6xHisGFP and not to non-specific interactions between GFP and the avidin-coated magnetic beads, a plot using the same protocol was constructed with 6xHisGFP (FIG. 4) instead of the b-6xHisGFP. This resulted in a straight line at high fluorescence signal, indicating that no binding to the avidin-coated beads is occurring, and thus verifying the specificity of the b-6xHisGFP label.

Thus, GFP has been successfully used as a label for the development of a heterogeneous binding assay for biotin. A competitive binding assay involving GFP as the label and the feasibility of using GFP for quantitative analytical applications are demonstrated. The availability of GFP mutants with enhanced fluorescence characteristics such as higher emission efficiencies, faster folding rates, and Stokes shifts optimized for particular regions of the spectrum should also afford in the development of novel applications for this protein. In addition, the preparation of fusion proteins through the N-terminus of GFP and the ability to express the protein in a variety of cell types will further allow for the possibility of more sensitive assays for multi-analyte, and in vivo measurements.

Fusion Protein Assay

In this study, GFP has also been employed as a quantitative label in the development of an immunoassay for a polypeptide. GFP possesses several desirable characteristics for potential use as a quantitative label. Homogeneous, one-to-one populations of conjugates with GFP can be produced through genetic engineering methods, which is not possible with synthetic fluorescent compounds. Also, mutations can be introduced into the protein to create mutants with specifically desired properties. For example, recent mutants of GFP are many times brighter than the wild type protein [Yang, T. et al. (1996) Nucleic Acids Res. 24: 4592-4593; Cormack, B. et al. (1996) Gene 173: 33-38]. Other mutations have produced mutants with modified spectral properties like the enhanced blue fluorescent protein (EBFP). Cloning vectors containing the EBFP gene, which are suitable as a source of the gene or for fusing a heterologous protein to the C- or N-terminus of EBFP, are commercially available from CLONTECH.

For this study, the octapeptide, Asp-Tyr-Lys-Asp-Asp-Asp-Asp-Lys (SEQ ID NO: 3), was chosen as a model analyte. This octapeptide was chosen to demonstrate that fusion proteins between GFP and small peptides can be constructed without loss of the protein's fluorescence properties. Another reason for choosing this peptide was due to its commercial availability and its sequence, which contains six carboxylic groups and three amino groups. The presence of these functional groups would make it difficult, if not impossible, to prepare a homogeneous population of conjugates. The production of a heterogeneous population could seriously impair the ability of such a small peptide to bind to its corresponding antibody. By using fused-gene technology, we have obtained a homogeneous population of mono-substituted conjugates of GFP and the octapeptide. This method allows for control of the placement of fluorescent label on the octapeptide, and provides high lot-to-lot reproducibility of the peptide-GFP conjugate population.

To produce the octapeptide-GFP conjugates, an expression vector, pSD100 (FIG. 5), was constructed as previously described using the DNA sequence for the wild-type GFP (10). After expression in E. coli, the product was purified using an Anti-Flag M2 affinity gel that can be used for both amino-terminal and carboxy-terminal Flag fusion proteins (23). The final product was a fusion protein where the C-terminal of the octapeptide was fused to the N-terminal GFP.

The purified octapeptide-GFP conjugate retained the same fluorescence excitation and emission spectrums as the native GFP. The excitation spectrum contained the characteristic maximum at 395 nm and minimum at 470 nm. The emission spectrum was also the same as the native protein with a maximum emission at 509 nm and a shoulder at 540 nm.

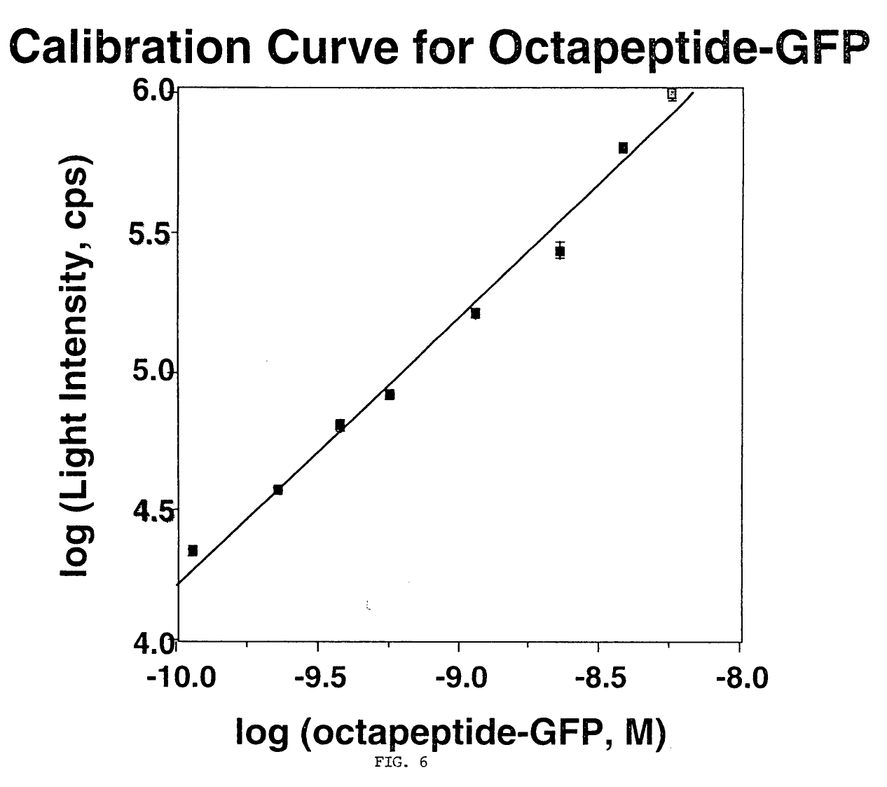

After determining the excitation and emission properties of the fusion protein, a calibration curve was constructed using the maximum excitation wavelength, 395 nm, and monitoring the fluorescence emission at 509 nm. The calibration plot for the octapeptide-GFP fusion protein is shown in FIG. 6. The fluorescence spectrum of the fusion protein has a linear range extending from 10⁻⁸ M to 10⁻¹⁰ M. For the development of the heterogeneous immunoassay, this curve was then used to select a starting concentration of the conjugate for the subsequent steps in the assay development. Since the assay is a competitive, or a limited reagent method in which free analyte and labeled analyte compete for binding sites on the monoclonal antibody (M2 antibody) (24), the choice of labeled-analyte concentration affects the obtainable detection limits for the assay. This concentration was chosen to be as low as possible while still maintaining a signal to be measured that was well above the background signal.

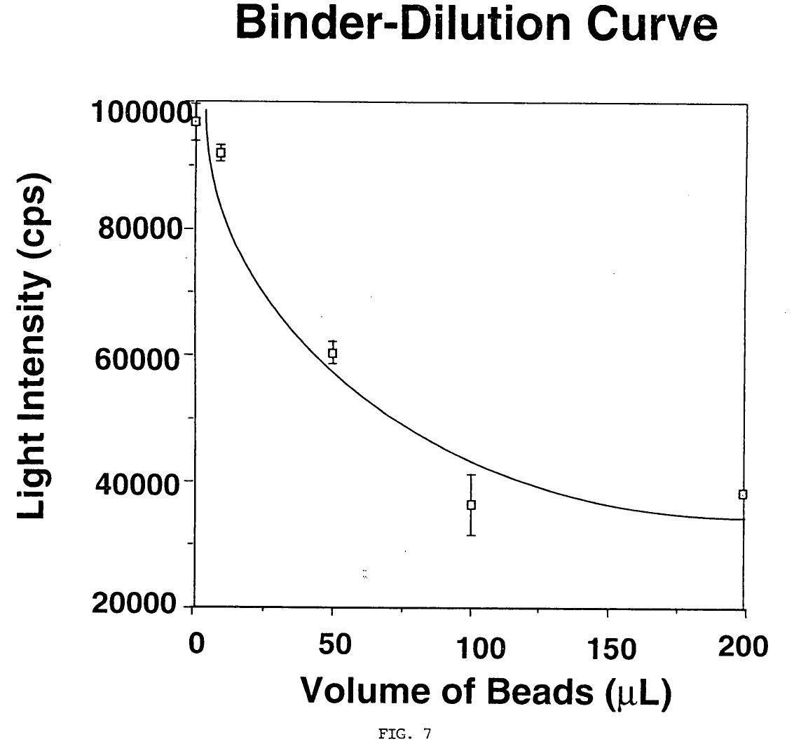

Having selected the conjugate concentration, the next step in the assay development was to study the interaction between the octapeptide-GFP and the anti-octapeptide M2 monoclonal antibody. The M2 antibody had been immobilized on agarose beads using a hydrazide linkage (25). Due to possible scattering problems, all subsequent measurements of fluorescence emission were made from the liquid rather than the solid phase. To determine initially whether the octapeptide would still bind to the M2 antibody, and the optimum amount of the solid phase to use for the assay, a binder-dilution curve was constructed. This was done by incubating a fixed concentration of the octapeptide-GFP with varying concentrations of the immobilized antibody for 90 minutes. After incubation, the tubes were centrifuged and the liquid phase (approximately 1.7 mL) was pipetted out carefully into disposable cuvettes for measurement. The resulting curve is shown in FIG. 7, and it can been seen that as the amount of binder increases, the fluorescence emission decreases until the curve becomes essentially flat. The flat portion of the curve represents the immobilized antibody concentration at which all the labeled-analyte that can bind to the antibody is bound. In order to establish a competition between free and labeled-analyte, free antibody sites must still be available, so this portion of the curve is avoided for selection of the binder concentration. The amount of antibody-beads for the dose-response curve was chosen from the linear portion of the curve where the signal is still changing as the binder concentration changes. The selection from this region was such that a minimum amount of antibody was used while the fluorescence emission was still sufficient to perform the assay.

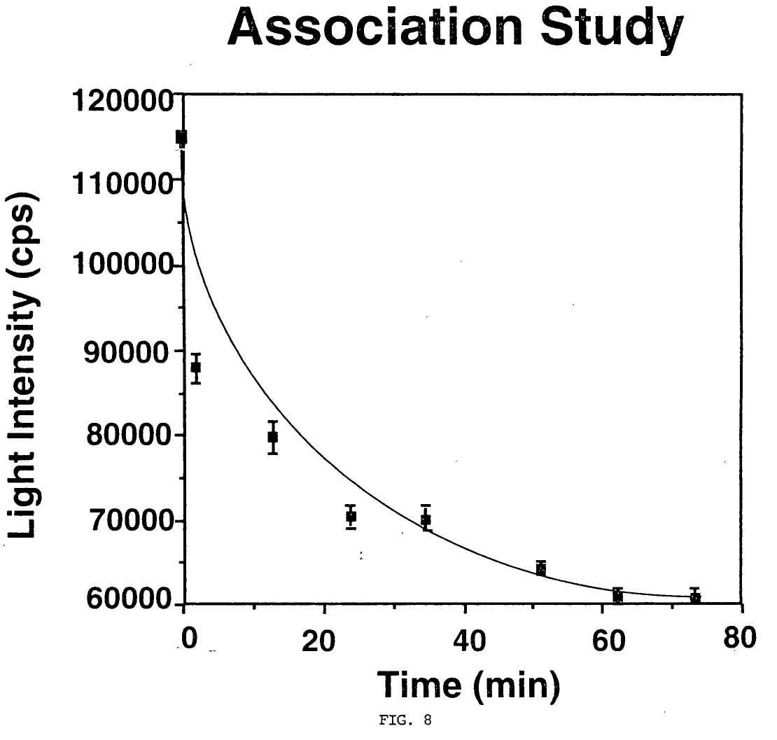

The above incubation time was used to ensure binding, however, this time must also be optimized. A time study was performed by incubating 200 μL of 140 μg/mL of the immobilized M2 antibody suspension with 100 μL of 1.14×10⁻⁸ M of the octapeptide-GFP in 1.7 mL of buffer for increasing amounts of time. The resulting curve is shown in FIG. 8, and from this curve 25 min was selected as sufficient time for the incubation steps of the assay. No substantial increase in binding was observed after this time.

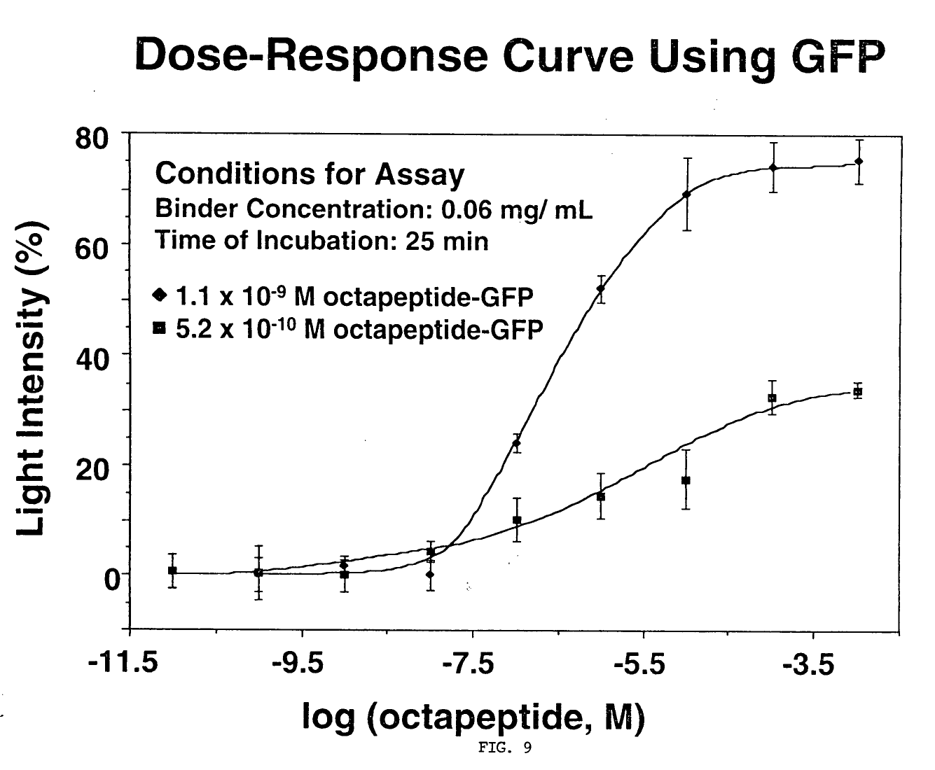

Once the optimum parameters were determined, dose-response curves were then constructed. The dose-response curves were generated in a sequential manner incubating different amounts of the free octapeptide with the immobilized antibody, followed by a centrifugation and washing step before incubating the antibody with a set amount of octapeptide-GFP conjugate. The solution was then centrifuged to extract the liquid phase for measurement. FIG. 9 shows two sigmoidal curves, one in which approximately 200 μL of 1.14×10⁻⁸ M of the octapeptide-GFP was used, and the other using only 90 μL of the same solution. The curves were normalized to percent light intensity, with the signal being lower at lower free octapeptide concentrations. As the amount of free octapeptide increases, more becomes bound to the solid phase, resulting in the displacement of more of the fluorescently labeled-octapeptide into the liquid, which is measured. The portion of the curve, which is not flat, but has a slope, corresponds to the analytically useful part of the curve. It can be seen that decreasing the amount of labeled analyte shifts the curve to lower detection limits. The detection limit for the octapeptide was determined at 1.0×10⁻⁸ M for the octapeptide in the sample using a signal-to-noise ratio of 3.

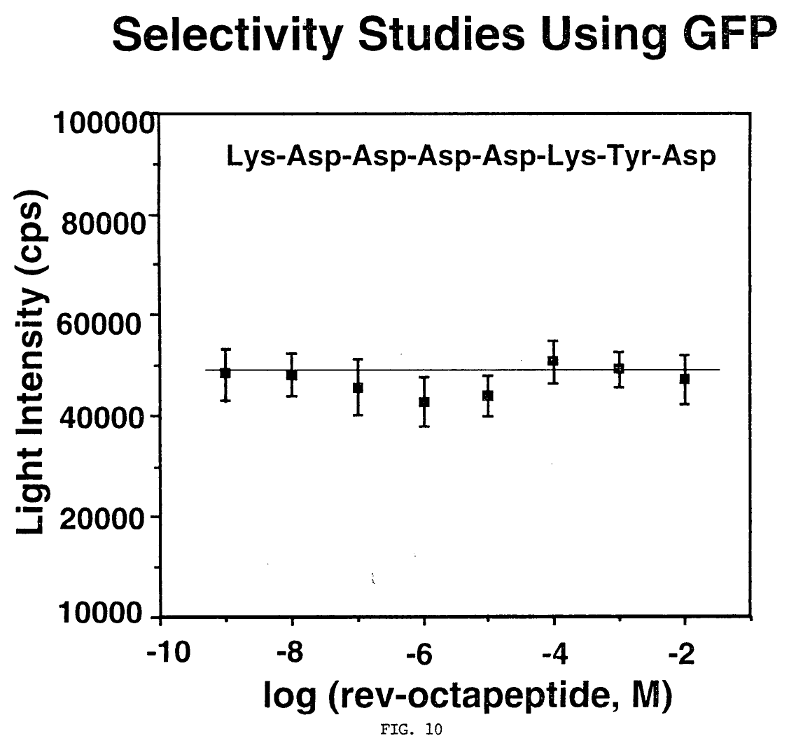

Another dose-response curve was constructed to test the selectivity of the assay. For this curve, the rev-octapeptide was used, which gave essentially no response (FIG. 10). This result was expected since epitope mapping has shown that the M2 antibody selectively recognizes the sequence Asp-Tyr-Lys-Xaa-Xaa-Asp-Xaa-Xaa-Xaa (SEQ ID NO: 4)(where Xaa can be any amino acid residue) (26). The rev-octapeptide differs in the first three critical amino acids.

In conclusion, we have used GFP as a quantitative label for the development of a heterogeneous immunoassay for a polypeptide. To our knowledge, the use of GFP in this respect has not been previously examined.

The present invention has been described with reference to certain examples for purposes of clarity and understanding. It should be appreciated that certain obvious modifications and improvements of the invention can be practiced without departing from the spirit and scope of the appended claims and their equivalents.

### REFERENCES

The pertinent disclosures of the references discussed above are incorporated herein by reference.
1. Ward, W. et al. (1982) Photochem. Photobiol. 35, 803-808.
2. Morise, H. et al. (1974) Biochemistry 13, 2656-2662.
3. Niwa, H. et al. (1996) Proc. Natl. Acad. Sci. USA 93, 13617-13622.
4. Reid, B. G. et al. (1997) Biochemistry 36, 6786-6791.
5. Ormo, M. et al. (1996) Science 273, 1392-1395.
6. Chalfie, M. (1995) Photochem. Photobiol. 62, 651-656.
7. Prasher, D. et al. (1992) Gene 111, 229-233.
8. Inouye, S. et al. (1994) FEBS Lett. 341, 277-280.
9. Cubitt, A. et al. (1995) TIBS 20, 448-455.
10. Chalfie, M. et al. (1994) Science 263, 802-805.
11. Hentz, N. et al. (1995) Anal. Chem. 67, 1014-1018.
12. Kronick, M. et al. (1983) Clin. Chem. 29, 1582-1586.
13. Kronick, M. et al. (1986) J. Immunol. Methods 92, 1-13.
14. Kim, B. et al. (1990) Anal. Chem. 62, 2663-2668.
15. Porath, J: (1992) Protein Expression Purif. 3, 263-281.
16. Bayer, E. et al. (1990) Methods Enzymol. 184, 138-160.
17. Bachas, L. et al. (1986) Anal. Biochem. 156, 223-238.
18. Daunert, S. et al. (1989) Anal. Chem. 61, 2160-2164.
19. Witkowski, A. et al. (1993) Anal. Chem. 65, 1147-1151.
20. Krull, I. et al. (1997) LC-GC 15, 534-539.
21. Barbarakis, M. et al. (1992) Bioconjugate Chem. 2, 225-229.
22. Barbarakis, M. et al. (1993) Anal. Chem. 65, 457-460
23. Hopp, T. et al. (1988) Biotechnology 6, 1205-1210.
24. Diamandis, E. et al. Eds. Immunoassay, Academic Press: San Diego, Calif. 1996.
25. Brizzard, B. et al. (1994) Biotechniques 16, 730-735.
26. Noren, K. et al. (1996) NEB Transcript 8, 3.
27. Kohler and Milstein, Nature, 256: 495-497 (1975)
28. Maniatis, T., Fritsch, D. F., Sambrook, J., Eds. Molecular Cloning A Laboratory Manual; Cold Spring Harbor Laboratory Press: Cold Spring Harbor, N.Y., 1989.
   Mutant GFP References:
28. Handfield M, et al., “ASD-GFP vectors for in vivo expression technology in Pseudomonas aeruginosa and other gram-negative bacteria,” Biotechniques, (1998) 24(2): 261-264.
29. Coxon A, et al. “Proteins that glow in green and blue,” Chem Biol. (1995) 2(3): 119-121.
30. Miyawaki A, et al., “Fluorescent indicators for Ca2+ based on green fluorescent proteins and Calnodulin,” Nature, (1997) 388(6645): 882-887.
31. Dickson R., et al. “On/off blinking and switching behaviour of single molecules of green fluorescent protein,” Nature, (1997), 388(6640): 355-358.
32. Reid B., et al., “Chromophore formation in green fluorescent protein,” Biochemistry, (1997) 36(22): 6786-6791.
33. Bierhuizen M., et al. “Green fluorescent protein variants as markers of retroviral-mediated gene transfer in primary hematopoietic cells and cell lines,” Biochem Biophys Res Commun. (1997) 234(2): 371-375.
34. Wu C, et al., “Novel green fluorescent protein (GFP) baculovirus expression vectors,” Gene, (1997) 190(1): 157-162.
35. Swaminathan R., et al. “Photobleaching recovery and anisotropy decay of green fluorescent protein GFP-S65T in solution and cells: cytoplasmic viscosity probed by green fluorescent protein translational and rotational diffusion,” Biophys J. (1997) 72(4): 900-1907.
36. Brejc K, et al., “Structural basis for dual excitation and photoisomerization of the Aequorea victoria green fluorescent protein,” Proc Natl Acad Sci USA, (1997) 94(6): 2306-2311.
37. Kimata Y, et al., “A novel mutation which enhances the fluorescence of green fluorescent protein at high temperatures,” Biochem Biophys Res Commun. (1997) 232(1): 69-73.
38. Kohler R., et al. “The green fluorescent protein as a marker to visualize plant mitochondria in vivo,” Plant J. (1997) 11(3): 613-621.
39. Siemering K., et al. “Mutations that suppress the thermosensitivity of green fluorescent Protein,” Curr Biol. (1996) 6(12): 1653-1663.
40. Yang T., et al. “Optimized codon usage and chromophore mutations provide enhanced sensitivity with the green fluorescent protein,” Nucleic Acids Res. (1996) 24(22): 4592-4593.
41. Anderson M., et al. “Simultaneous fluorescence-activated cell sorter analysis of two distinct transcriptional elements within a single cell using engineered green fluorescent proteins,” Proc Natl Acad Sci USA, (1996) 93(16): 8508-8511.
42. Zylka M., et al. “Optimized filter set and viewing conditions for the S65T mutant of GFP in living cells,” Biotechniques, (1996) 21(2): 220-221.
43. Oker-Blom C, et al. “Highly efficient production of GFP and its derivatives in insect cells for visual in vitro applications,” FEBS Lett. (1996) 389(3): 238-243.
44. Ropp J., et al. “Aequorea green fluorescent protein: simultaneous analysis of wild-type and blue-fluorescing mutant by flow cytometry,” Cytometry, (1996) 24(3): 284-288.
45. Reichel C., et al. “Enhanced green fluorescence by the expression of an Aequorea victoria green fluorescent protein mutant in mono- and dicotyledonous plant cells,” Proc Natl Acad Sci USA. (1996) 93(12): 5888-5893.
46. Heim R., et al. “Engineering green fluorescent protein for improved brightness, longer wavelengths and fluorescence resonance energy transfer,” Curr Biol. (1996) 6(2): 178-182.
47. Cormack B., et al. “FACS-optimized mutants of the green fluorescent protein (GFP)” Gene, (1996) 173(1 Spec No): 33-38.
48. Ehrig T, et al. “Green-fluorescent protein mutants with altered fluorescence excitation Spectra,” FEBS Lett. (1995) 367(2): 163-166.
49. Heim R., et al. “Wavelength mutations and posttranslational autoxidation of green fluorescent protein,” Proc Natl Acad Sci USA. (1994) 91(26): 12501-12504.
50. Yang, T. et al., “Quantitative Comparison of Three Red-Shifted GFP Variants,” CLONTECHniques January 1997 (IF73441B)
51. “Living Colors EYFP Vectors” October 1997, CLONTECHniques XII(4):14-15
52. “New Living Colors GFP Vectors”, July 1996, CLONTECHniques XI(3):20-22.

1. A method of determining the amount of an analyte in a fluid sample, comprising:
(a) conjugating said analyte with a ligand to create a sample comprising a ligand-analyte conjugate;
(b) contacting the sample of the ligand-analyte conjugate with a solution containing a ligand-green fluorescent protein conjugate and an immobilized anti-ligand in a predefined ratio, wherein the ligand is identical in said ligand-analyte conjugate and said ligand-green fluorescent protein conjugate, and said ligand-analyte conjugate and said ligand-green fluorescent protein conjugate competitively bind with said anti-ligand;
(c) separating said immobilized anti-ligand and any conjugates binding thereto from any unbound conjugates to create a supernatant containing unbound conjugates;
(d) measuring the intensity of fluorescence of said immobilized anti-ligand or said supernatant; and
(e) relating the measured intensity of fluorescence to the amount of analyte in the sample.

2. The method of claim 1, wherein said ligand is biotin and said anti-ligand is avidin or streptavidin.

3. The method of claim 1, wherein said ligand is a hapten and said anti-ligand is an immunoglobulin that specifically binds said hapten.

4. The method of claim 1, wherein said analyte is a biomolecule.

5. The method of claim 4, wherein said biomolecule is selected from the group consisting of agonists, antagonists, toxins, venoms, viral epitopes, hormones, hormone receptors, polypeptides, enzymes, cofactors, enzyme substrates, drugs, lectins, sugars, oligonucleotides, oligosacharides, proteins, and antibodies.

6. A method of determining the amounts of at least two analytes in a fluid sample, comprising:
(a) preparing a sample of at least two analyte-ligand conjugates by conjugating a first analyte with a first ligand to create a first analyte-ligand conjugate, and
conjugating at least a second analyte with at least a second ligand to create at least a second analyte-ligand conjugate;
(b) contacting the sample comprising the first ligand-analyte conjugate and at least second ligand-analyte conjugate with a solution containing (i) a first ligand-first green fluorescent protein conjugate and a first immobilized anti-ligand in a predefined ratio, and (ii) at least a second ligand-second green fluorescent protein conjugate, and at least a second immobilized anti-ligand in a predefined ratio, wherein said first ligand-analyte conjugate and said first ligand-first green fluorescent protein conjugate competitively bind with said first anti-ligand, but do not bind with said at least second anti-ligand, said at least second ligand-analyte conjugate and said at least second ligand-second green fluorescent protein conjugate competitively bind with said at least second anti-ligand, but do not bind with said first anti-ligand, and said first ligand-first green fluorescent protein conjugate emits fluorescent light which is distinct from fluorescent light emitted by said at least second ligand-second fluorescent protein conjugate;
(c) separating said immobilized first and at least second anti-ligand and any conjugates binding thereto from any unbound conjugates to create a supernatant containing unbound conjugates;
(d) distinctly measuring the intensity of fluorescence of bound or unbound first ligand-first green fluorescent protein conjugate and the intensity of fluorescence of bound or unbound at least second ligand-second fluorescent protein conjugate; and
(e) relating the measured intensity of fluorescence of said bound or unbound first ligand-first green fluorescent protein conjugate and the intensity of fluorescence of bound or unbound at least second ligand-second fluorescent protein conjugate to the amount of first and at least second analyte, respectively, in the sample.

7. The method of claim 6, wherein said first and at least second analytes are biomolecules.

8. The method of claim 7, wherein said biomolecules are selected from the group consisting of agonists, antagonists, toxins, venoms, viral epitopes, hormones, hormone receptors, polypeptides, enzymes, cofactors, enzyme substrates, drugs, lectins, sugars, oligonucleotides, oligosaccharides, proteins, and antibodies.

9. The method of claim 1, in which the step (d) of measuring the fluorescence intensity is performed by evanescent wave fluorimetry.

10. The method of claim 6, in which the step (d) of distinctly measuring the fluorescence intensity distinctly measuring of the first ligand-first green fluorescent protein conjugate and the fluorescence intensity of the at least second ligand-second fluorescent protein conjugate is performed by evanescent wave fluorimetry.
